(19) United States
(12) Patent Application Publication
(10) Pub. No.: US 2020/0187685 A1
(43) Pub. Date: Jun. 18, 2020

(54) AUTOMATICALLY HEIGHT-ADJUSTABLE PILLOW

(71) Applicant: MAETEL CO., LTD., Seoul (KR)

(72) Inventors: Key JEONG, Suwon-si (KR); Seung Yo KIM, Seoul (KR); Woo June CHOI, Incheon (KR); Je Hui LEE, Incheon (KR)

(73) Assignee: MAETEL CO., LTD., Seoul (KR)

(21) Appl. No.: 16/796,338

(22) Filed: Feb. 20, 2020

Related U.S. Application Data
(63) Continuation of application No. PCT/KR2018/008496, filed on Jul. 27, 2018.

(30) Foreign Application Priority Data
Aug. 21, 2017 (KR) 10-2017-0105241

(51) Int. Cl. A47G 9/10; A61F 5/56

(52) U.S. Cl. CPC A47G 9/1027; A61F 5/56; A47G 9/1081

(57) ABSTRACT

A pillow for automatically adjusting a height of the pillow is provided. The pillow includes a pillow cover including a pressure sensor to sense pressure applied when a user puts a head of the user on the pillow, a plurality of air pockets positioned inside the pillow cover, an air injection unit to adjust an amount of air inside the air pocket, and a controller. The control unit senses pressure of a contact region between a body of the user and the pillow cover when the user puts the head of the user on the pillow, and controls the air injection unit to adjust the air amount inside at least one of the plurality of air pockets such that distribution of the pressure of the contact region is within a preset range, when the distribution of the pressure of the contact region is out of the preset range.

### CROSS-REFERENCE TO RELATED APPLICATIONS

The present application is a continuation of International Patent Application No. PCT/KR2018/008496, filed on Jul. 27, 2018 which is based upon and claims the benefit of priority to Korean Patent Application No. 10-2017-0105241 filed on Aug. 21, 2017. The disclosures of the above-listed applications are hereby incorporated by reference herein in their entirety.

### BACKGROUND

Embodiments of the inventive concept described herein relates to a pillow for automatically adjusting the height thereof.

Recently, as the interest in the sleeping quality has increased and the number of patients having a turtle neck syndrome has increased due to the prolonged use of a computer, pillows that induce a correct sleeping posture, such as cervical pillows, have increased. However, since the patients have different body types, the use of the same pillow by all users has a limitation in producing the effect of the cervical pillow. Therefore, there is a necessity to provide a pillow to be matched to the body type of the user and the steeping state of the user.

[Prior Art] Korean Unexamined Patent Publication No. 10-2016-0125840 (published on Nov. 1, 2016).

### SUMMARY

Embodiments of the inventive concept provide a pillow having an automatically adjustable height.

The objects of the inventive concept are not limited to the above, but other effects, which are not mentioned, will be apparently understood to those skilled in the art.

According to an exemplary embodiment, a pillow for automatically adjusting the height thereof includes a pillow cover including a pressure sensor to sense pressure applied when a user puts a head of the user on the pillow, a plurality of air pockets positioned inside the pillow cover, an air injection unit to adjust an amount of air inside the air pocket, and a controller. The control unit senses pressure of a contact region between a body of the user and the pillow cover when the user puts the head of the user on the pillow, and controls the air injection unit to adjust the air amount inside at least one of the plurality of air pockets such that distribution of the pressure of the contact region is within a preset range, when the distribution of the pressure of the contact region is out of the preset range.

The details of the inventive concept are included in the detailed description and accompanying drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 illustrates a schematic perspective view, a perspective view, and an exploded perspective view of a pillow for automatically adjusting the height thereof, according to an embodiment of the inventive concept;

FIG. 2 is a block diagram illustrating the configuration of a pillow for automatically adjusting the height thereof, according to an embodiment of the inventive concept;

FIG. 3 is a view illustrating a method for extracting a signal corresponding to a snore, according to an embodiment of the inventive concept;

FIGS. 4A and 4B illustrate examples of the sleeping posture of the user using a pillow for automatically adjusting the height thereof, according to an embodiment of the inventive concept;

FIG. 5 illustrates another example of a sleeping posture of the user using a pillow for automatically adjusting the height thereof, according to an embodiment of the inventive concept;

FIGS. 6A and 6B illustrate still another examples of a sleeping posture of the user using a pillow for automatically adjusting the height thereof, according to an embodiment of the inventive concept;

FIGS. 7A-7C illustrate sleep record feedbacks provided to a user based on data obtained from a pillow for automatically adjusting the height thereof, according to an embodiment of the inventive concept; and

FIG. 8 illustrates an example of a sleeping managing service provided to a user based on data obtained from a pillow for automatically adjusting the height thereof, according to an embodiment of the inventive concept.

### DETAILED DESCRIPTION

Advantage points and features of the inventive concept and a method of accomplishing thereof will become apparent from the following description with reference to the following figures, wherein embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. The inventive concept may be defined by scope of the claims. Meanwhile, the terminology used herein to describe embodiments of the invention is not intended to limit the scope of the inventive concept.

The terminology used in the inventive concept is provided for the illustrative purpose, but the inventive concept is not limited thereto. As used herein, the singular terms “a,” “an” and “the” are intended to include the plural forms as well, unless the context clearly indicates otherwise. Furthermore, it will be further understood that the terms “comprises”, “comprising,” “includes” and/or “including”, when used herein, specify the presence of stated elements, steps, operations, and/or devices, but do not preclude the presence or addition of one or more other components, steps, operations and/or devices. The same reference numerals will be assigned to the same component throughout the whole specification, and “and/or” refers to that components described include not only individual components, but at least one combination of the components. It will be understood that, although the terms “first”, “second”, etc., may be used herein to describe various components, these components should not be limited by these terms. These terms are only used to distinguish one component from another component. Thus, a first component to be described below may be a second component without departing from the teachings of the inventive concept.

Unless otherwise defined, all terms (including technical and scientific terms) used herein have the same meaning as commonly understood by those skilled in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Spatially relative terms, such as “below”, “beneath”, “lower”, “above”, “upper”, and the like, may be used herein to make it easier to describe the relationship between one component and another component. It will be understood that the spatially relative terms are intended to encompass different orientations of components in use or operation in addition to the orientation depicted in the figures. For example, when a device illustrated in accompanying drawings is reversed, a device provided ‘below’ or ‘beneath’ another device may be placed ‘above’ another device. Accordingly, the term “below” may include both concepts of “below” and “above. A device may be oriented in a different direction. Accordingly, terminology having relatively spatial concepts may be variously interpreted depending on orientations.

Hereinafter, exemplary embodiments of the inventive concept will be described with reference to accompanying drawings.

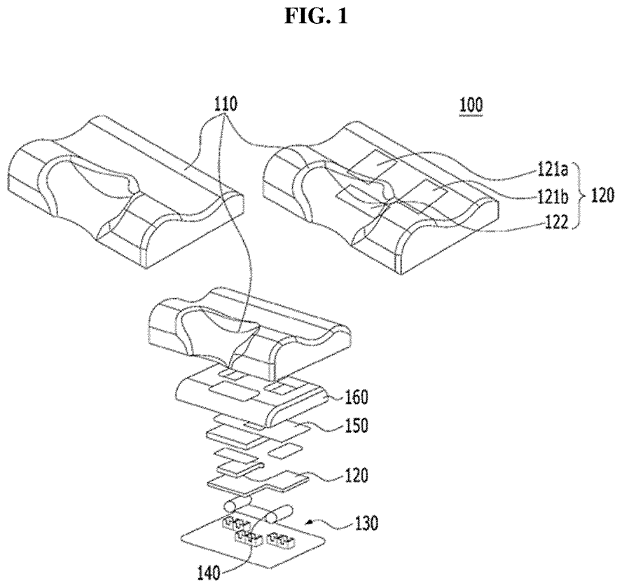

FIG. 1 illustrates a schematic perspective view, a perspective view, and an exploded perspective view of a pillow for automatically adjusting the height thereof, according to an embodiment of the inventive concept.

Referring to FIG. 1, a pillow 100 for automatically adjusting the height thereof includes a pillow cover 110 configured to sense pressure applied when a user puts the head of the user on the pillow 100, a plurality of air pockets 120 positioned inside the pillow cover 110, an air injection unit 130 to adjust an amount of air inside the air pocket 120, and a control unit 140. The pillow 100 may further include at least one of a rubber cover 150 and an upper cover 160 interposed between the air pocket 120 and the pillow cover 110 to prevent a user from sensing an internal part including the air pocket 120 and to prevent the driving noise of a motor included in the air injection unit 130. The rubber cover 150 and the upper cover 160 may maintain the basic thickness of the pillow 100, and the upper cover 160 may fix various internal parts.

The pillow cover 110 serves as a region to make contact with the body of the user when the user puts the head of the user on the pillow 100, and surrounds internal parts of the pillow 100. The pillow cover 110 may include a pressure sensor to sense the position and size of the pressure applied when the user puts the head of the user on the pillow 100. The pressure sensor may be included, as a component, in the pillow cover 110 or may be positioned inside the pillow cover 110. For example, the pillow cover 110 may include conductive fabric, and the pressure sensor may be a fabric sensor that is able to sense the pressure obtained by converting electrical data applied when the user puts the head of the user on the pillow 100, but the inventive concept is not limited thereto. For example, the control unit 140 may measure the pressure, which is applied to the contact region by sensing the variation of the potential made from the pressure applied to the contact region with the body of the user when the user puts the head of the user on the pillow 100, by using the pillow cover 110 including the fabric sensor. The pillow cover 110 may be formed of a flexible material having a specific thickness.

The pillow cover 110 may have a concave shape at a portion corresponding to the position of the neck when the user puts the head of the user on the pillow 100. Accordingly, the body shape of the user may be reflected and the stable support may be made until the upper portion of a shoulder of the user.

The plurality of air pockets 120 may be positioned inside the pillow cover 110. The plurality of air pockets 120 may be linked to each other or spaced apart from each other using a material the same as or similar to a housing material of the air pockets.

The air pockets 120 are positioned inside the pillow cover 110 to form the height (thickness) of the pillow 100. According to an embodiment, the air pockets 120 may include at least two of an air pocket 121a positioned corresponding to one side portion of the head, an air pocket 121b positioned corresponding to an opposite side portion of the head, an air pocket 122 positioned corresponding to the neck of the user, and an air pocket positioned corresponding to the central portion or the upper portion of the head, when the user puts the head of the user on the pillow 100.

The following description of the present embodiment will be made while focusing on the air pocket 120 including the air pocket 121a positioned corresponding to one side portion of the head, the air pocket 121b positioned corresponding to an opposite side portion of the head, and the air pocket 122 positioned corresponding to the neck of the user. For the convenience of explanation, the air pockets 121a and 121b positioned corresponding to opposite side portions of the head may be referred to as side air pockets, and the air pocket 122 positioned corresponding to the neck may be referred to as a cervical air pocket.

Air is injected into each of the plurality of air pockets 120, and the volumes of the air pockets 120 may be adjusted by adjusting an air amount injected therein. The air amount of each of the air pockets 120 may be independently adjusted.

The air injection unit 130 may adjust an amount of air inside the air pocket 120 by injecting air into the air pocket 120 or discharging air from the inner part of the air pocket 120. For example, the air injection unit 130 may include an air compressor including a motor, a pump, an air pressure sensor, an air tank. The air injection unit 130 may determine an air amount by measuring the air pressure inside the air pocket 120 using an air pressure sensor, and may adjust the air amount to be injected into or discharged out of the air pocket 120 under the control of the control unit 140.

The control unit 140 may measure the pressure applied to the pillow cover 110 using data sensed by the pressure sensor, and may control the air injection unit 120 to adjust the air amount of the air pocket based on the measured pressure.

According to an embodiment, the control unit 140 may determine a sleeping posture of the user, a sleeping quality of the user, or a steeping state of the user, and the height of the pillow 100 may be adjusted by controlling the air amount inside the air pocket 120 by controlling the air injection unit 130 to improve a sleeping situation of the user based on the determination result,

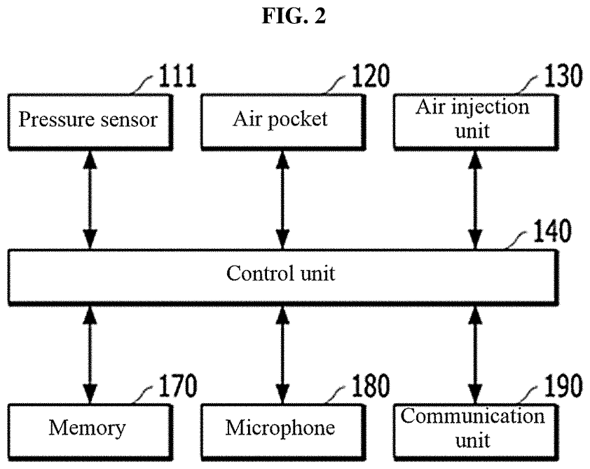

FIG. 2 is a block diagram illustrating the configuration of the pillow 100 for automatically adjusting the height thereof, according to an embodiment of the inventive concept. Hereinafter, the same module as the module described with reference to FIG. 1 will be described with the same reference numeral assigned to the module in FIG. 1.

Referring to FIG. 2, the pillow 100 for automatically adjusting the height thereof may include the pressure sensor 111 that is able to sense the pressure applied when the user puts the head of the user on the pillow 100, the plurality of air pockets 120, the air injection unit 130 to adjust the air amount injected into the air pocket 120, the control unit 140, a memory 170, a microphone 180, and a communication unit 190. According to an embodiment, at least two of the above-described modules may be integrally implemented with one module.

The memory 170 stores a program necessary to detect the sleeping posture or the steeping state of the user putting the head of the user on the pillow 100, and stores a program to adjust the air amount of the air pocket 120 to improve the sleeping quality of the user based on the detected sleeping posture and/or the steeping state.

The control unit 140 may detect the sleeping posture of the user and the steeping state of the user using at least one of pressure sensed by the pressure sensor 111, the amount of air injected into the air pocket 120, information obtained through the microphone 180, and information obtained from the outside through the communication unit 190, and may adjust the air amount of the air pocket 120 to improve the sleeping quality.

The microphone 180 may receive an external sound signal and convert the external sound signal to an electrical signal. The sound signal received in the microphone 180 may include surrounding noise in addition sleeping noise (representatively, a snore) of the user. The control unit 140 may analyze the electrical signal obtained through the microphone 180 to extract the electrical signal corresponding to the snore of the user except for a sound corresponding to the surrounding noise.

The memory 170 may store information about a frequency, a waveform, amplitude, or a cycle of the snore of a user or a general person.

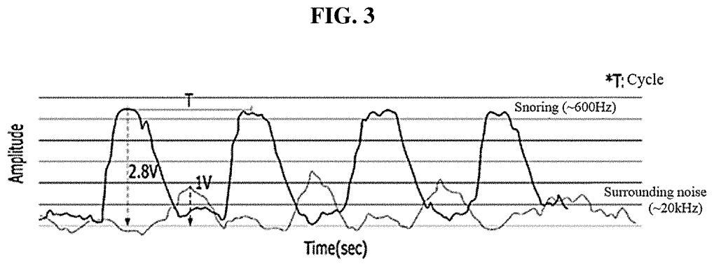

FIG. 3 is a view illustrating a method for extracting a signal corresponding to a snore, according to an embodiment of the inventive concept.

FIG. 3 illustrates the waveform of an electrical signal corresponding to the external sound obtained through the microphone 180. For example, the control unit 140 may allow the electric signal obtained through the microphone 180 to pass through the low pass filter to remove the signal corresponding to the surrounding noise, thereby filtering only the electrical signal having the frequency corresponding to the snore. In addition, the control unit 140 may determine whether the user is currently snoring and the intensity of the snore, by using the information on the cycle, the waveform, or the amplitude of the electrical signal corresponding to the snore previously stored in the memory 170.

Referring back to FIG. 2, the communication unit 190 may transmit data to or receive the data from an external device/server through a wireless communication scheme.

For example, the control unit 140 may transmit information on a sleeping posture, or a steeping state of the user to a mobile phone of the user or the external server through the communication unit 190. Accordingly, the mobile phone of the user or the external server may record and analyze the sleeping quality of the user to provide necessary information to the user.

For another example, the control unit 140 may transmit the electrical signal, which is obtained through the microphone 180, to the external device or server through the communication unit 190 instead of detecting, by the pillow 100, whether the user snores or the snoring degree, and may receive the analysis result of the snoring from the external device or server.

For another example, when the pillow 100 is not provided with the microphone 180, the control unit 140 may obtain an electrical signal using a microphone provided in the external device (for example, the mobile phone of the user) that internetworks with the pillow 100 and may analyze the snoring of the user based on the electrical signal. In addition, the control unit 140 of the pillow 100 may receive and use only the analysis result of the snoring of the user from the external device.

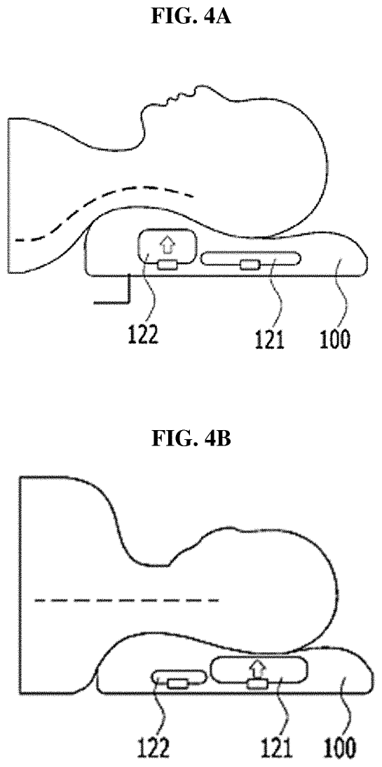

The following description will be made with reference to FIGS. 4 to 6 regarding the height of the pillow 100 automatically adjusted depending on the sleeping posture or the steeping state of the user.

FIGS. 4A and 4B illustrate the sleeping postures of the user by using the pillow 100 for automatically adjusting the height thereof, according to an embodiment of the inventive concept.

FIGS. 4A and 4B illustrate the cross-sectional views taken along a Y axis of the pillow 100 for automatically adjusting the height thereof, according to an embodiment of the inventive concept.

The control unit 140 may sense the pressure of the contact region between the body of the user and the pillow cover 110 when the user puts the head of the user on the pillow 100, and may control the air injection unit 130 to adjust the air amount of the air pocket 120 depending on a preset reference based on the distribution of the sensed pressure of the contact region.

For example, as illustrated in FIG. 4A, it is assumed that a user puts the head of the user on the pillow 100 while lying on the back of the user. The neck of a person has the shape of alphabet ‘C’, and the back of a head more protrudes. Accordingly, the empty space may be made between the pillow 100 and the neck. Especially, when a user has a thicker shoulder, a thicker back, or has a back of the head, which significantly protrudes, the larger empty space may be formed between the pillow 100 and the neck. In this case, the pillow 100 fails to uniformly support the neck and the head of the user. Accordingly, a forward head posture may be formed or excessive pressure may be formed in a portion of a spine.

For another example, as illustrated in FIG. 4B, when the user puts the head of the user on the pillow 100 while lying on the side of the user, the head of the user presses the pillow cover 110 with the greater pressure and the large empty space is formed between the neck of the user and the pillow 100, so the spin may be bent down.

Accordingly, the control unit 140 may determine the sleeping posture of the user based on the distribution of the pressure applied to the pillow cover 110 when the user puts the head of the user on the pillow 100. For example, the control unit 140 may determine the sleeping posture such as a posture in which the neck of the user is not sufficiently supported by the pillow 100 or the posture in which the chin of the user is bent toward the chest of the user as the pillow 100 is excessively high, when the user puts the head of the user on the pillow 100 and when the distribution of pressure applied to the pillow cover 110 is out of the preset range. In addition, the control unit 140 may control the air injection unit 130 to adjust the air amount of at least one of the air pockets 120 such that the pressure applied to the pillow cover 110 is uniformly distributed in a preset range.

Referring to FIG. 4A, when the user puts the head of the user on the pillow 100 while lying on the back, the control unit 140 increases the volume by injecting a specific amount of air into the cervical air pocket 122 based on the pressure sensing result. Accordingly, the pillow 100 sufficiently supports the neck of the user such that the C-shape of the neck is comfortably maintained.

In addition, referring to FIG. 4B, when the user lies on the side of the user, the control unit 140 injects a specific amount of air into two side air pockets 121 based on the pressure sensing result to increase the volumes of the air pockets 121, such that the head of the user may be comfortably supported without sagging down.

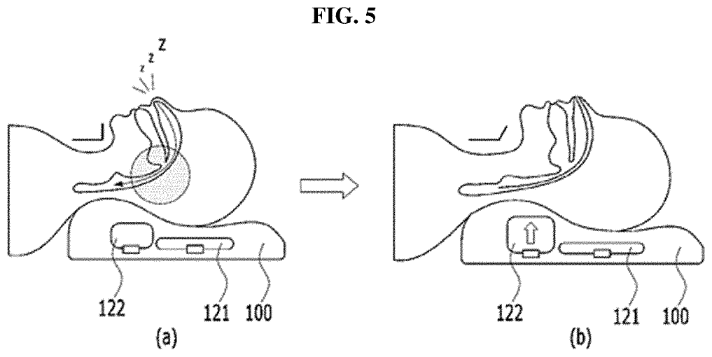

FIG. 5 illustrates another example of a sleeping posture of the user using a pillow for automatically adjusting the height thereof, according to an embodiment of the inventive concept.

FIG. 5 illustrates an embodiment of improving the snoring of the user when the snoring of the user is sensed. Whether the user snores, or the snoring degree may be determined inside the pillow 100, or may be determined by the external device or server internetworking with the pillow 100 such that the determining result may be transmitted to the pillow 100, as described above with reference to FIGS. 2 and 3.

The control unit 140 may control the air injection unit 130 to adjust the amount of the air in the cervical air pocket 122 by using the intensity of the electrical signal corresponding to the snoring of the user and the sensing result of the pressure applied to the pillow cover 110.

Referring to (a) of FIG. 5, the control unit 140 may recognize that the user is snoring when the intensity of the electrical signal corresponding to the snoring of the user exceeds a first reference which is preset. In addition, the control unit 140 may detect the sleeping posture (for example, the user lies on the back of the user) of the user, based on the sensing result of the pressure applied to the pillow cover 110.

In addition, as illustrated in (b) of FIG. 5, the control unit 140 may control the air injection unit 130 such that the air amount of the cervical air pocket 122 is increased to a preset level. When the volume of the cervical air pocket 122 is increased in the state the user lies on the back of the user, the back of the head of the user is bent back and the throat of the user is open so the user comfortably takes a break. Accordingly, the snoring of the user is improved. In this case, the volume of the side air pocket 122 is uniformly maintained and may be reduced by a preset degree.

The control unit 140 may continuously monitor the intensity of the electrical signal corresponding to the snoring of the user in the state that the volume of the cervical air pocket 122 is maintained as illustrated in (b) of FIG. 5.

The control unit 140 may determine that the snoring of the user is stopped and return the state of the pillow 100 to the state as in (a) of FIG. 5 to prevent the neck of the user from being stressed, when the intensity of the electrical signal corresponding to the snoring becomes equal to or lower than a second reference lower than the first reference.

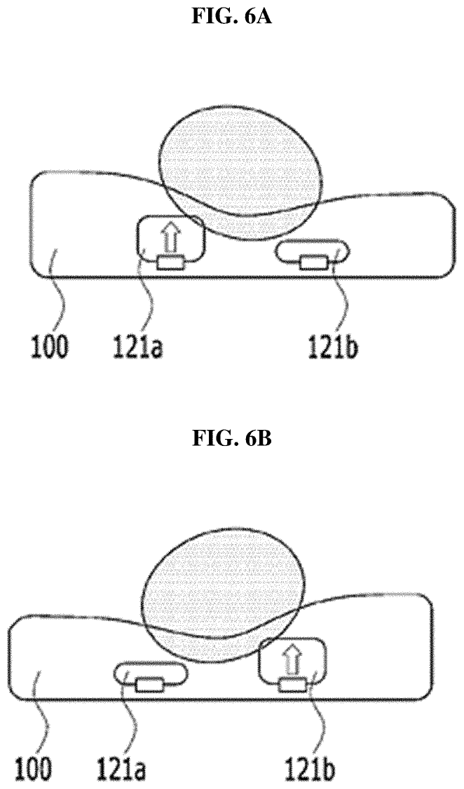

Alternatively, when the intensity of the electrical signal corresponding to the snoring is not equal to or lower than the second reference, the control unit 140 may more maintain the posture of the user to the posture as in (b) of FIG. 5, or may change the posture of the user by adjusting the volume of the air pocket as describe below with reference to FIGS. 6A and 6B.

FIGS. 6A and 6B illustrate still another examples of a sleeping posture of the user using a pillow for automatically adjusting the height thereof, according to an embodiment of the inventive concept.

FIGS. 6A and 6B illustrate the cross-sectionals view taken along a Y axis of the pillow 100 for automatically adjusting the height thereof, according to an embodiment of the inventive concept.

The control unit 140 may control the air injection unit 130 to adjust the air amount in the side air pocket 121 by using the intensity of the electrical signal corresponding to the snoring of the user and the sensing result of the pressure applied to the pillow cover 110.

The control unit 140 may recognize that the user is snoring when the intensity of the electrical signal corresponding to the snoring of the user exceeds a third reference which is preset. In addition, the control unit 140 may detect the sleeping posture (for example, the user lies on the back of the user) of the user, based on the sensing result of the pressure applied to the pillow cover 110. In this case, the third reference may be lower than or higher than the first reference and is higher than the second reference.

The control unit 140 may increase the air amount of any one 121a or 121b of two air pockets 121a and 121b positioned corresponding to opposite side portions of the head when the user puts the head of the user on the pillow 100. In this case, the control unit 140 may maintain or reduce the air amount of a remaining one of the two air pockets 121a and 121b. In other words, when the control unit 140 increases the volume of any one of the two side air pockets 121a and 121b, the head of the user faces the side such that the snoring of the user may be improved.

According to an embodiment, when the snoring fails to be improved or the snoring degree exceeds a preset degree from the beginning even if the height of the pillow 100 is adjusted as described above, the control unit 140 may control the air injection unit 130 to alternately increase or decrease the air amount of each of the two side air pockets 121a and 121b at preset time intervals (see (a) of FIG. 5 and (b) of FIG. 5). When the head of the user is inclined to one side for a long time, the neck of the user may be stressed. Accordingly, the control unit 140 may alternately adjust opposite heights of the pillow 100, so the snoring may be improved within the extent that the neck is not stressed.

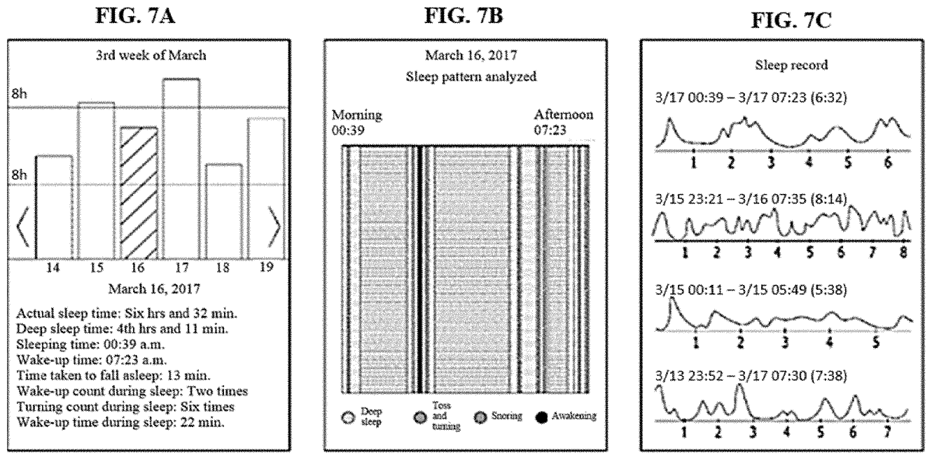

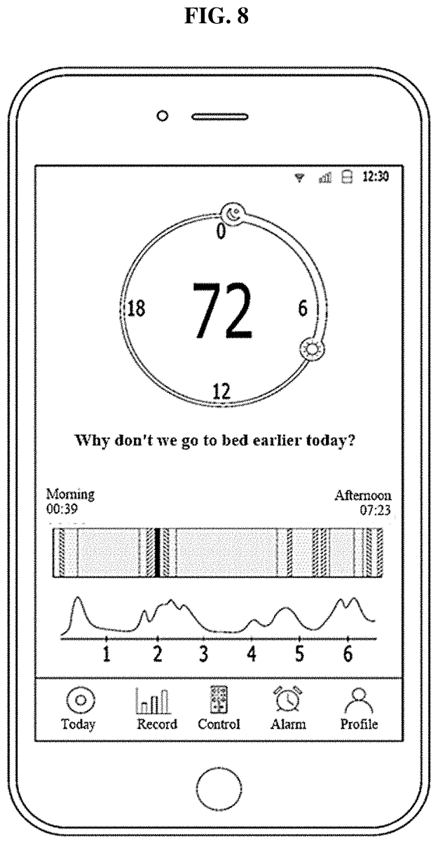

The following description will be made with reference to FIGS. 7 and 8 regarding an embodiment of providing a sleep record feedback or a sleeping managing service to a user, based on sensed data from the pillow 100 and snoring data analyzed by the pillow 100 or the external device/server.

FIGS. 7A-7C illustrate sleep record feedbacks provided to a user based on data obtained from a pillow for automatically adjusting the height thereof, according to an embodiment of the inventive concept; and

The control unit 140 of the pillow 100 may control the communication unit 190 to transmit data, which corresponds to the pressure sensed through the pressure sensor 111, to the external device (for example, the portable phone of the user) or the external server. In addition, when the control unit 140 is able to analyze the electrical signal corresponding to the snoring of the user, the control unit 140 may control the communication unit 190 to transmit data, which corresponds to the analysis result, to the external device or the external server. In this case, the external server may be a server of a service provider who operates the sleeping improving service.

The external service or server may analyze sleeping data of a sample user, learn the sleeping pattern through a machining learning scheme, analyze data received from the pillow 100 using the learned data, and generate a sleep record feedback of the user based on the received data.

A user may access a mobile application installed in a cellular phone or a tablet PC, a program installed in a desktop computer or a laptop computer, or a web-site to see the sleep record feedback of the user. The mobile application, the problem, or the web-site may be provided by the operator of the server.

The sleep record feedback may include a daily sleeping time, a sleeping start time, a sleeping end time, a sleeping quality, or a snoring analysis result. The external device or server may analyze the pressure sensing result included in data received from the pillow 100 to analyze the daily sleeping time, the sleeping start time, the sleeping end time, or the sleeping quality, and may analyze the snoring time, the snoring degree, or the sleeping quality based on snoring analysis data.

FIG. 8 illustrates an example of a sleeping managing service provided to a user based on data obtained from a pillow for automatically adjusting the height thereof, according to an embodiment of the inventive concept.

Referring to FIGS. 7A-7C, the external device or server may provide a user customized sleeping managing service based on the sleep record of the user.

Referring to FIG. 8, the external device or server suggests a correct sleeping habit to the user based on the sleep record of the user, or may provide the sleeping managing service such as an advice of a proper sleeping environment based on a current temperature or humidity.

As described above, according to the embodiment of the inventive concept, the pillow 100 for automatically adjusting the height thereof is provided based on the sleeping posture of the user, or the sleeping habit of the user, thereby improving the sleeping quality of the user.

The method or the algorithm steps described regarding the embodiment of the inventive concept may be implemented in hardware, and implemented with a software module executed by the hardware, or the combination of the software and the hardware. A soft module may reside in a random access memory (RAM), a read only memory (ROM), an erasable programmable ROM (EPROM), an electrically erasable programmable ROM (EEPROM), a flash memory, a hard disk, a removable disk, a CD-ROM, or a computer readable recording medium well known in the art to which the inventive concept pertains.

Although embodiments of the inventive concept have been described with reference to accompanying drawings those skilled in the art should understand that various modifications are possible without departing from the technical scope of the inventive concept or without changing the technical sprite or the subject matter of the inventive concept. Therefore, those skilled in the art should understand that the technical embodiments are provided for the illustrative purpose in all aspects and the inventive concept is not limited thereto.

As described above, embodiments of the inventive concept may provide a pillow for automatically adjusting the height thereof to be matched to the body type and the steeping state of a user who puts the head of the user on the pillow.

The objects of the inventive concept are not limited to the above, but other effects, which are not mentioned, will be apparently understood to those skilled in the art.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A pillow for automatically adjusting a height of the pillow, the pillow comprising:
a pillow cover including a pressure sensor to sense pressure applied when a user puts a head of the user on the pillow;
a plurality of air pockets positioned inside the pillow cover;
an air injection unit to adjust an amount of air inside the air pocket; and
a controller,
wherein the control unit is configured to:
sense pressure of a contact region between a body of the user and the pillow cover when the user puts the head of the user on the pillow; and
control the air injection unit to adjust the air amount inside at least one of the plurality of air pockets such that distribution of the pressure of the contact region is within a preset range, when the distribution of the pressure of the contact region is out of the preset range.

2. The pillow of claim 1, wherein the pillow cover includes conductive fabric, and
wherein the pressure sensor includes a fabric sensor that is able to sense pressure obtained by converting electrical data applied when the user puts the head of the user on the pillow.

3. The pillow of claim 1, wherein the plurality of air pockets includes:
at least two of an air pocket positioned corresponding to one side portion of the head, an air pocket positioned corresponding to an opposite side portion of the head, an air pocket positioned corresponding to a neck of the user, and an air pocket positioned corresponding to an upper portion of the head, when the user puts the head of the user on the pillow.

4. The pillow of claim 3, further comprising:
a microphone to receive an external sound signal and to convert the external sound signal to an electrical signal,
wherein the microphone obtains an electrical signal corresponding to a snore of the user, and
wherein the control unit controls the air injection unit to adjust the air amount inside the air pocket, based on an intensity of the electrical signal.

5. The pillow of claim 4, wherein the control unit controls the air injection unit to increase the air amount inside the air pocket, which is positioned corresponding to the neck, to be a preset level when the user puts the head of the user on the pillow, when the intensity of the electrical signal exceeds a first reference.

6. The pillow of claim 5, wherein the control unit controls the air injection unit to decrease the air amount in the air pocket, which is positioned corresponding to the neck, when the intensity of the obtained electrical signal corresponding to the snore of the user is equal to or lower than a second reference lower than the first reference, after increasing the air amount inside the air pocket, which is positioned corresponding to the neck, to be the preset level.

7. The pillow of claim 4, wherein the control unit controls the air injection unit to increase one of two air pockets positioned corresponding to opposite side portions of the head, respectively, when the user puts the head of the user on the pillow, and to decrease a remaining one of the two air pockets, when the intensity of the electrical signal exceeds a third reference.

8. The pillow of claim 7, wherein the control unit controls the air injection unit to alternately increase or decrease, at a preset time interval, air amounts inside the two air pockets positioned corresponding to the opposite side portions of the head, respectively, when the user puts the head of the user on the pillow, when the intensity of the electrical signal exceeds the third reference.

9. The pillow of claim 4, further comprising:
a communication unit to transmit, to an external terminal, data corresponding to an electrical signal corresponding to a sleeping noise, which is obtained from the microphone, of the user,
wherein the communication unit receives analysis data of a snoring state of the user from the external terminal, and
wherein the control unit controls the air injection unit to adjust the air amount inside the air pocket, based on the received analysis data.

10. The pillow of claim 9, wherein the control unit controls the communication unit to transmit, to the external terminal, the data corresponding to the pressure sensed through the pressure sensor, during sleep of the user.
